(19) United States
(12) Patent Application Publication
(10) Pub. No.: US 2022/0370657 A1
(43) Pub. Date: Nov. 24, 2022

(54) RECORDING DEVICE

(71) Applicant: SEIKO EPSON CORPORATION, Tokyo (JP)

(72) Inventor: Koki ISHIKAWA, Matsumoto-shi (JP)

(21) Appl. No.: 17/664,110

(22) Filed: May 19, 2022

(30) Foreign Application Priority Data
May 19, 2021 (JP) 2021-084518

(51) Int. Cl. A61L 2/10; A61L 2/24; B41J 29/17

(52) U.S. Cl. CPC A61L 2/10; A61L 2/24; B41J 29/17; A61L 2202/14; A61L 2202/20

(57) ABSTRACT

A recording device includes: a medium accommodation portion configured to accommodate a plurality of media; a feeding portion configured to send out the media from the medium accommodation portion; a recording section configured to perform recording on the media sent out from the medium accommodation portion; an ultraviolet-light emitting portion provided at a position opposed to the medium accommodation portion and configured to emit ultraviolet light toward the media accommodated in the medium accommodation portion; and a control unit configured to control the ultraviolet-light emitting portion to emit ultraviolet light to the media by utilizing a period before the feeding portion starts to feed the media.

The present application is based on, and claims priority from JP Application Serial Number 2021-084518, filed on May 19, 2021, the disclosure of which is hereby incorporated by reference herein in its entirety.

### BACKGROUND

### 1. Technical Field

The present disclosure relates to a recording device configured to perform recording on a medium.

### 2. Related Art

There is an inkjet printer serving as one example of a recording device. The inkjet printer discharges ink serving as one example of a liquid onto a recording sheet serving as one example of a medium, to perform recording.

In recent years, there are users' needs for removing bacteria or virus attached on the recording sheet. In relation to the users' needs described above, JP-A-2005-115268 proposes an image forming device that performs a sterilization process to a medium. This image forming device includes an ultraviolet light lamp provided at a position upstream of a photoreceptor drum in a transport path for the medium, and performs the sterilization process to the medium.

In the configuration described in JP-A-2005-115268, in a case where the ultraviolet light lamp is used to perform the sterilization process to the medium, it is necessary to temporarily stop transporting the medium or slow down the transport velocity at the time of performing the sterilization process in order to reliably obtain a sterilization effect. This makes it more likely to reduce a printing throughput.

### SUMMARY

A recording device according to the present disclosure that solves the problem described above includes a medium accommodation portion configured to accommodate a plurality of media, a feeding portion configured to send out the media from the medium accommodation portion, a recording section configured to perform recording on the media sent out from the medium accommodation portion, an ultraviolet-light emitting portion provided at a position opposed to the medium accommodation portion and configured to emit ultraviolet light toward the media accommodated in the medium accommodation portion, and a control unit configured to control the ultraviolet-light emitting portion to emit ultraviolet light to the media by utilizing a period before the feeding portion starts to feed the media.

In addition, a recording device according to the present disclosure includes a medium accommodation portion configured to accommodate a plurality of media, the medium accommodation portion being attachable to and detachable from a device body, a feeding portion configured to send out the media from the medium accommodation portion, a recording section configured to perform recording on the medium sent out from the medium accommodation portion, an ultraviolet-light emitting portion configured to emit ultraviolet light toward the media, an attached state detector configured to detect an attached state of the medium accommodation portion, and a control unit configured to control the ultraviolet-light emitting portion, the control unit being configured to, when an elapsed time from a time when the medium accommodation portion is set in the attached state is a first period, cause a period, in which the second ultraviolet-light emitting portion emits ultraviolet light to the media, to be shorter than when the period is a second period that is shorter than the first period.

In addition, a recording device according to the present disclosure includes a medium accommodation portion configured to accommodate a plurality of media and be attachable to and detachable from a device body, a feeding portion configured to send out the media from the medium accommodation portion, a recording section configured to perform recording on the media sent out from the medium accommodation portion, an ultraviolet-light emitting portion configured to emit ultraviolet light toward the media, a stack height detector configured to detect a stack height of the media in the medium accommodation portion, and a control unit, in which the control unit is configured to control the ultraviolet-light emitting portion so as to, when the stack height is a first height, cause a period, in which the ultraviolet-light emitting portion emits ultraviolet light to the media, to be shorter than when the stack height is a second height that is lower than the first height.

In addition, a recording device according the present disclosure includes a liquid discharging head configured to discharge a liquid to a medium to perform recording, a first transport region disposed downstream of a recording section in a transport path configured to transport the medium, the first transport region being configured to transport the media at a reduced transport velocity of the media, an ultraviolet-light emitting portion provided at the first transport region and configured to emit ultraviolet light toward the medium, and a control unit configured to control the ultraviolet-light emitting portion and configured to control the ultraviolet-light emitting portion so as to emit the ultraviolet light to the medium that passes through the first transport region.

In addition, a recording device according to the present disclosure includes a recording section configured to perform recording on a medium, a switch-back path disposed downstream of the recording section in a transport path configured to transport the medium, the switch-back path being configured to perform switch-back transport of the medium, an ultraviolet-light emitting portion disposed at an entrance to the switch-back path and configured to emit ultraviolet light toward the medium, and a control unit configured to control the ultraviolet-light emitting portion, the control unit being configured to control the ultraviolet-light emitting portion so as to emit ultraviolet light to the medium when the medium enters the switch-back path and when the medium exits the switch-back path.

In addition, a recording device according to the present disclosure includes a liquid discharging head configured to discharge a liquid to a medium to perform recording, in which the liquid discharging head is provided so as to move in a width direction that is a direction intersecting a medium transport direction, an ultraviolet-light emitting portion configured to emit ultraviolet light toward the media is disposed at a position that is opposable to the media when the liquid is discharged by the liquid discharging head, and a control unit configured to control the ultraviolet-light emitting portion is configured to control the ultraviolet-light emitting portion so as to emit ultraviolet light to the media when the liquid is discharged by the liquid discharging head.

### BRIEF DESCRIPTION OF THE DRAWINGS

FIG. 1 is a diagram illustrating an entire configuration of a recording device.

FIG. 2 is a diagram illustrating a medium transport path in a printer portion.

FIG. 3 is a diagram illustrating a wire used to guide a medium at a position of a UV lamp.

FIG. 4 is a block diagram illustrating a control system relating to the UV lamp.

FIG. 5 is a flowchart showing a first control example of emission of ultraviolet light to a medium.

FIG. 6 is a flowchart showing a second control example of emission of ultraviolet light to a medium.

FIG. 7 is a flowchart showing a third control example of emission of ultraviolet light to a medium.

FIG. 8 is a flowchart showing a fourth control example of emission of ultraviolet light to a medium.

### DESCRIPTION OF EXEMPLARY EMBODIMENTS

Below, the present disclosure will be schematically described.

A recording device according to a first aspect includes: a medium accommodation portion configured to accommodate a plurality of media; a feeding portion configured to send out the media from the medium accommodation portion; a recording section configured to perform recording on the media sent out from the medium accommodation portion; an ultraviolet-light emitting portion provided at a position opposed to the medium accommodation portion and configured to emit ultraviolet light toward the media accommodated in the medium accommodation portion; and a control unit configured to control the ultraviolet-light emitting portion to emit ultraviolet light to the media by utilizing a period before the feeding portion starts to feed the media.

With the present aspect, the recording device includes the ultraviolet-light emitting portion configured to emit ultraviolet light toward the media accommodated in the medium accommodating portion, and the control unit configured to control the ultraviolet-light emitting portion is configured to control the ultraviolet-light emitting portion so as to emit the ultraviolet light to the media by utilizing a period before the feeding portion starts to feed the media. This makes it possible to prevent the recording throughput from reducing in association with ultraviolet-light irradiation.

In the first aspect, a second aspect is configured such that, in a case of feeding a plurality of the media, the control unit sets the ultraviolet-light emitting portion in an emitting state before a first one of the media starts to be fed, and then, the emitting state is kept at least until feeding of a last one of the media is completed.

With the present aspect, in a case of feeding a plurality of the media, the control unit sets the ultraviolet-light emitting portion in the emitting state before the first one of the media starts to be fed, and then, the emitting state is kept at least until feeding of the last one of the media is completed. This makes it possible to prevent the recording throughput from reducing in association with ultraviolet-light irradiation while securing the period of time for emitting the ultraviolet light to the medium.

In the first or second aspect, a third aspect is configured such that the control unit sets the ultraviolet-light emitting portion in an emitting state upon a power supply for the device being turned on.

With the present aspect, upon the power supply for the device being turned on, the control unit sets the ultraviolet-light emitting portion in the emitting state. Thus, it is possible to quickly start to feed the media upon receiving an instruction to start to perform recording. This makes it possible to prevent the recording throughput from reducing in association with ultraviolet-light irradiation.

In any one of the first to third aspects, a fourth aspect is configured such that the medium accommodation portion is attachable to and detachable from a device body including the recording section, and the device body includes: an attached state detector configured to detect an attached state of the medium accommodation portion; and a second ultraviolet-light emitting portion configured to emit ultraviolet light toward the media that are transported, the ultraviolet-light emitting portion being a first ultraviolet-light emitting portion, in which the control unit is configured to, when an elapsed period from a time when the medium accommodation portion is set in the attached state is a first period, cause a period, in which the second ultraviolet-light emitting portion emits ultraviolet light to the media, to be shorter than when the period is a second period that is shorter than the first period.

With the present aspect, the second ultraviolet-light emitting portion is provided. Thus, by utilizing the second ultraviolet-light emitting portion, it is possible to more reliably perform a sterilization process to the media.

Here, bacteria or virus attached on the media die over time, and the entire number thereof reduces, accordingly. Thus, the period of time for emitting the ultraviolet light can be set to be short. On the basis of such a property, the present aspect is configured such that the media are irradiated with the second ultraviolet-light emitting portion for a period of time, and this period of time is set to be shorter when an elapsed period from a time when the medium accommodation portion changes into the attached state is the first period, than when the elapsed period is the second period that is shorter than the first period. Thus, it is possible to further prevent the recording throughput from reducing in association with ultraviolet-light irradiation.

In any one of the first to third aspects, a fifth aspect is configured such that the device body includes: a stack height detector configured to detect a stack height of the media in the medium accommodation portion; and a second ultraviolet-light emitting portion configured to emit ultraviolet light toward the media that are transported, the ultraviolet-light emitting portion being a first ultraviolet-light emitting portion, in which the control unit is configured to, when the stack height is a first height, cause a period, in which the second ultraviolet-light emitting portion emits ultraviolet light to the media, to be shorter than when the stack height is a second height that is lower than the first height.

With the present aspect, the second ultraviolet-light emitting portion is provided. Thus, by utilizing the second ultraviolet-light emitting portion, it is possible to more reliably perform a sterilization process to the media.

Here, bacteria or virus attached on the media die over time, and the entire number thereof reduces, accordingly. Thus, the period of time for emitting ultraviolet light can be set to be short. On the basis of the property described above, the present aspect is configured such that, when the stack height is a first height, the control unit causes a period, in which the second ultraviolet-light emitting portion emits ultraviolet light to the media, to be shorter than when the stack height is a second height that is lower than the first height. In other words, the period of time for emitting ultraviolet light is set to be shorter with decrease in the number of sheets of the media in the medium accommodation portion. This makes it possible to further prevent the recording throughput from reducing in association with ultraviolet-light irradiation.

In the fourth or fifth aspect, a sixth aspect is configured such that a feed tray for setting and feeding the media one by one is provided, and the control unit uses an equal period as an emission time in which the second ultraviolet-light emitting portion emits ultraviolet light to the media fed from the feed tray.

The feed tray is a feed tray in which the media are set one by one and are fed. Thus, the period of time elapsed from a time when a user touches the media that have been set at the feed tray is short, and it is preferable to secure the period of time for emitting ultraviolet light. On the basis of the property as described above, the present aspect is configured such that the control unit uses an equal period as an emission time for irradiating, with ultraviolet light, the media that have been fed from the feed tray. This makes it possible to appropriately perform a sterilization process to the media that have been fed from the feed tray.

In any one of the first to third aspects, a seventh aspect is configured such that the recording section includes a liquid discharging head configured to discharge a liquid to the media to perform recording, a first transport region is disposed downstream of the recording section in a transport path configured to transport the media, the first transport region being configured to transport the media at a reduced transport velocity of the media, a second ultraviolet-light emitting portion is provided in the first transport region and is configured to emit ultraviolet light toward the media, the ultraviolet-light emitting portion being a first ultraviolet-light emitting portion, and the control unit controls the second ultraviolet-light emitting portion so as to emit ultraviolet light to the media that pass through the first transport region.

With the present aspect, the second ultraviolet-light emitting portion is provided. Thus, it is possible to more reliably perform the sterilization process to the media, as compared with the configuration in which only the first ultraviolet-light emitting portion is provided.

In addition, in a case of the recording device configured to discharge a liquid to the media to perform recording, a dry waiting period may be set for a transport path configured to transport the media in order to dry the liquid. The dry waiting period can be created by reducing the transport velocity of the media. This reduction in the transport velocity includes setting the transport velocity to be at zero, that is, includes stopping transporting the media. By utilizing such a property, the present aspect is configured such that the second ultraviolet-light emitting portion is provided in a first transport region configured to reduce the transport velocity of the media to transport the media, that is, in a transport region for drying the media, and the second ultraviolet-light emitting portion is used to emit the ultraviolet light by utilizing the dry waiting period. This makes it possible to prevent the recording throughput from reducing in association with ultraviolet-light irradiation.

In any one of the first to third aspect, an eighth aspect is configured such that a switch-back path is provided downstream of the recording section in a transport path configured to transport the media, the switch-back path being configured to perform switch-back transport of the media, a second ultraviolet-light emitting portion configured to emit ultraviolet light toward the media is provided at an entrance to the switch-back path, the ultraviolet-light emitting portion being a first ultraviolet-light emitting portion, and the control unit controls the second ultraviolet-light emitting portion so as to emit ultraviolet light to the media when the media enter the switch-back path and when the media exit the switch-back path.

With the present aspect, the second ultraviolet-light emitting portion is provided. This makes it possible to more reliably perform the sterilization process to the media, as compared with the configuration in which only the first ultraviolet-light emitting portion is provided.

Furthermore, in the present aspect, the second ultraviolet-light emitting portion is provided at the entrance to the switch-back path, and the second ultraviolet-light emitting portion is used to irradiate the media with the ultraviolet light by using both the time when the media enter the switch-back path and the time when the media exit the switch-back path. This makes it possible to prevent the recording throughput from reducing in association with ultraviolet-light irradiation while securing the period of time for emitting ultraviolet light.

In any one of the first to third aspects, a ninth aspect is configured such that the recording section includes a liquid discharging head configured to discharge a liquid to the media to perform recording, the liquid discharging head is provided so as to be able to move in a width direction that is a direction intersecting a medium transport direction, a second ultraviolet-light emitting portion configured to emit ultraviolet light toward the media is provided at a position that is opposable to the media when the liquid is discharged by the liquid discharging head, the ultraviolet-light emitting portion being a first ultraviolet-light emitting portion, and the control unit controls the second ultraviolet-light emitting portion so as to emit ultraviolet light to the media when the liquid is discharged by the liquid discharging head.

With the present aspect, the second ultraviolet-light emitting portion is provided. This makes it possible to more reliably perform the sterilization process, as compared with the configuration in which only the first ultraviolet-light emitting portion is provided.

In addition, in the present aspect, the operation of discharging a liquid using the liquid discharging head and the process of sterilization of the media using the second ultraviolet-light emitting portion overlap with each other in terms of time. This makes it possible to prevent the recording throughput from reducing in association with ultraviolet-light irradiation.

A recording device according to a tenth aspect includes: a medium accommodation portion configured to accommodate a plurality of media and be attachable to and detachable from a device body; a feeding portion configured to send out the media from the medium accommodation portion; a recording section configured to perform recording on the media sent out from the medium accommodation portion; an ultraviolet-light emitting portion configured to emit ultraviolet light toward the media; an attached state detector configured to detect an attached state of the medium accommodation portion; and a control unit configured to control the ultraviolet-light emitting portion so as to, when an elapsed period from a time when the medium accommodation portion is set in the attached state is a first period, cause a period, in which the ultraviolet-light emitting portion emits ultraviolet light to the media, to be shorter than when the period is a second period that is shorter than the first period.

Bacteria or virus attached on the media die over time, and the entire number thereof reduces, accordingly. Thus, the period of time for emitting ultraviolet light can be set to be short. On the basis of such a property, the present aspect is configured to, when an elapsed period from a time when the medium accommodation portion is set in the attached state is a first period, cause a period, in which the ultraviolet-light emitting portion emits ultraviolet light to the media, to be shorter than when the period is a second period that is shorter than the first period. Thus, it is possible to further prevent the recording throughput from reducing in association with ultraviolet-light irradiation.

A recording device according to an eleventh aspect includes: a medium accommodation portion configured to accommodate a plurality of media and be attachable to and detachable from a device body; a feeding portion configured to send out the media from the medium accommodation portion; a recording section configured to perform recording on the media sent out from the medium accommodation portion; an ultraviolet-light emitting portion configured to emit ultraviolet light toward the media; a stack height detector configured to detect a stack height of the media in the medium accommodation portion; and a control unit, in which the control unit is configured to control the ultraviolet-light emitting portion so as to, when the stack height is a first height, cause a period, in which the ultraviolet-light emitting portion emits ultraviolet light to the media, to be shorter than when the stack height is a second height that is lower than the first height.

Bacteria or virus attached on the media die over time, and the entire number thereof reduces, accordingly. Thus, the period of time for emitting ultraviolet light can be set to be short. On the basis of such a property, the present aspect is configured such that, when the stack height is a first height, the control unit causes a period, in which the ultraviolet-light emitting portion emits ultraviolet light to the media, to be shorter than when the stack height is a second height that is lower than the first height. In other words, the period of time for emitting ultraviolet light is set to be shorter with decrease in the number of sheets of the media in the medium accommodation portion. This makes it possible to prevent the recording throughput from reducing in association with ultraviolet-light irradiation.

A recording device according to a twelfth aspect includes: a liquid discharging head configured to discharge a liquid to the media to perform recording; a first transport region disposed downstream of the recording section in a transport path configured to transport the media, the first transport region being configured to transport the media at a reduced transport velocity of the media; an ultraviolet-light emitting portion provided in the first transport region and configured to emit ultraviolet light toward the media; and a control unit configured to control the ultraviolet-light emitting portion, the control unit being configured to control the ultraviolet-light emitting portion so as to emit ultraviolet light to the media.

In a case of the recording device configured to discharge a liquid to the media to perform recording, a dry waiting period may be set for the transport path configured to transport the media in order to dry the liquid. The dry waiting period can be created by reducing the transport velocity of the media. This reduction in the transport velocity includes setting the transport velocity to be at zero, that is, includes stopping transporting the media. By utilizing such a property, the present aspect is configured such that the ultraviolet-light emitting portion is provided in a first transport region configured to reduce the transport velocity of the media to transport the media, that is, in a transport region for drying the media, and the ultraviolet-light emitting portion is used to emit the ultraviolet light by utilizing the dry waiting period. This makes it possible to prevent the recording throughput from reducing in association with ultraviolet-light irradiation.

A recording device according to a thirteenth aspect includes: a recording section configured to perform recording on a medium; a switch-back path provided downstream of the recording section in a transport path configured to transport the medium, the switch-back path being configured to perform switch-back transport of the medium; an ultraviolet-light emitting portion disposed at an entrance to the switch-back path and configured to emit ultraviolet light toward the medium; and a control unit configured to control the ultraviolet-light emitting portion, the control unit being configured to control the ultraviolet-light emitting portion so as to emit ultraviolet light to the medium when the medium enters the switch-back path and when the medium exits the switch-back path.

With the present aspect, the ultraviolet-light emitting portion configured to emit ultraviolet light toward the medium is provided at the entrance to the switch-back path, and ultraviolet light is emitted to the medium by utilizing both the time when the medium is sent to the switch-back path and the time when the medium exits the switch-back path. This makes it possible to prevent the recording throughput from reducing while securing the period of time for emitting ultraviolet light.

A recording device according to a fourteenth aspect includes a liquid discharging head configured to discharge a liquid to a medium to perform recording, in which the liquid discharging head is provided so as to be able to move in a width direction that is a direction intersecting a medium transport direction, an ultraviolet-light emitting portion configured to emit ultraviolet light toward the medium is provided at a position that is opposable to the medium when the liquid is discharged by the liquid discharging head, and the control unit configured to control the ultraviolet-light emitting portion is configured to control the ultraviolet-light emitting portion so as to emit ultraviolet light to the medium when the liquid is discharged by the liquid discharging head.

With the present aspect, the operation of discharging a liquid using the liquid discharging head and the process of sterilization of the medium using the second ultraviolet-light emitting portion overlap with each other. This makes it possible to prevent the recording throughput from reducing in association with ultraviolet-light irradiation.

In any one of the first to fourteenth aspects, a fifteenth aspect is configured such that, when a jam occurs in a transport path configured to transport the media, the control unit stops emitting ultraviolet light to the media.

With the present aspect, the cover configured to be able to open and close to expose an interior of the device is provided, and when a jam occurs in the transport path configured to transport the media, the control unit stops emitting the ultraviolet light to the media. This makes it possible to prevent the ultraviolet light from exerting an adverse effect on a user when the user opens the cover for the purposed of handling of the jam.

In any one of the first to fourteenth aspects, a sixteenth aspect includes a cover configured to open and close to expose the interior of the device, and an open-close detector configured to detect an open-close state of the cover, in which, upon detecting switching from a closed state of the cover to an opened state, the control unit stops emitting the ultraviolet light to the media.

The present aspect includes the cover configured to be able to open and close to expose the interior of the device, and the open-close detector configured to detect the open-close state of the cover, and the control unit stops emitting the ultraviolet light to the media upon detecting switching from the closed state of the cover to the opened state. This makes it possible to prevent the ultraviolet light from exerting an adverse effect on a user when the user opens the cover to access the interior of the device.

Below, the present disclosure will be specifically described.

The X-Y-Z coordinate system illustrated in each of the drawings is an orthogonal coordinate system. The X-axis direction indicates a device depth direction. The Y-axis direction indicates a device width direction. The Z-axis direction indicates a device height direction.

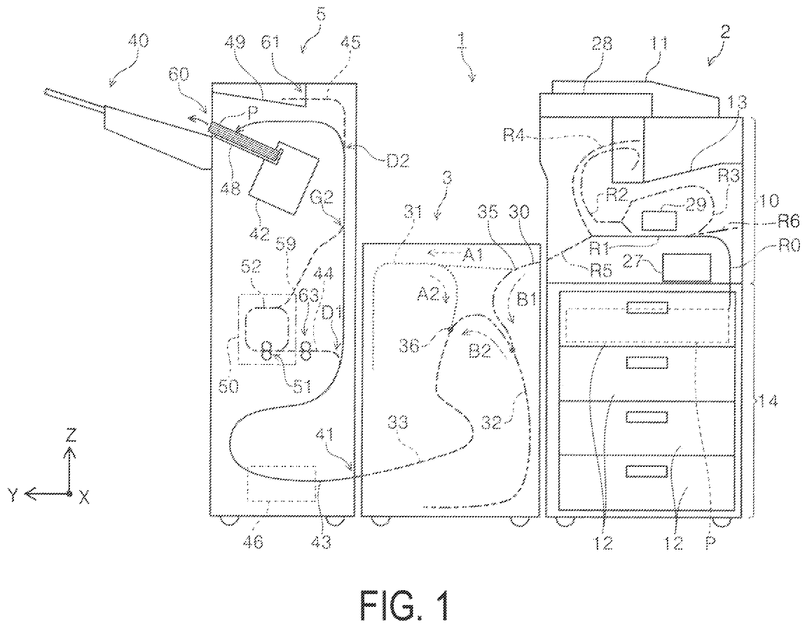

A recording device 1 illustrated in FIG. 1 includes a recording unit 2, an intermediate unit 3, and a post-processing unit 5 in order from right toward left of FIG. 1. Note that, in the recording device 1 according to the present embodiment, the recording unit 2, the intermediate unit 3, and the post-processing unit 5 are each configured as an independent device, and these devices are coupled to each other to constitute the recording device 1. Thus, the recording device according to the present embodiment can also be called a recording system or a liquid discharging system.

A control unit 27 configured to control various types of operations in the recording unit 2 is provided in the recording unit 2. The recording device 1 is configured such that the recording unit 2, the intermediate unit 3, and the post-processing unit 5 are mechanically and electrically coupled to each other, and are able to transport a medium from the recording unit 2 to the post-processing unit 5. In addition to the recording unit 2, the control unit 27 is able to control various types of operations in the intermediate unit 3 and the post-processing unit 5.

The recording unit 2 includes an operation unit 28, and is configured such that execution instructions and various types of setting concerning various types of processes in the recording unit 2, the intermediate unit 3, and the post-processing unit 5 can be inputted from the operation unit 28. In addition, the operation unit 28 includes a display panel (not illustrated), and is configured such that various types of information can be displayed at this display panel.

Note that, when an external computer (not illustrated) is coupled to the recording device 1, this external computer can be used to execute various types of settings and execution instructions that are similar to various types of settings and execution instructions performed at the operation unit 28.

The recording unit 2 is a device configured to perform recording on a medium that is transported. The intermediate unit 3 is a unit configured to receive, from the recording unit 2, the medium after recording to hand it over to the post-processing unit 5, and functions to invert the medium and also to promote drying of the medium. Note that the recording unit 2 also has a function of inverting the medium as well as a function of promoting drying of the medium, which will be described later.

The post-processing unit 5 includes: a hole-creating mechanism portion 46 configured to apply a hole creating process to the media; a drying portion 50 configured to apply a drying process to the medium; and an end binding portion 42 configured to perform an end binding process in which media are bundled together to bind the ends of the media.

Below, further description will be made of the recording unit 2, the intermediate unit 3, and the post-processing unit 5 in this order.

The recording unit 2 is configured as a multifunction machine including a printer section 10 configured to perform recording on a medium and a scanner section 11 that is an example of image capturing devices. In the embodiment, the printer section 10 is configured as a so-called inkjet type printer configured to eject ink, which is an example of recording liquid, onto a medium to perform recording.

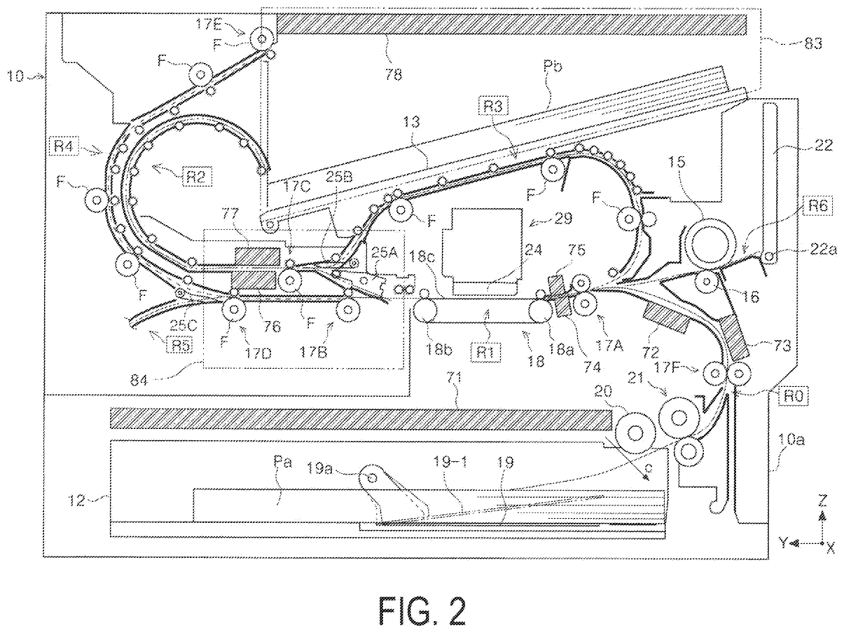

A plurality of medium accommodating cassettes 12 each serving as a medium accommodation portion configured to accommodate the medium are provided at a lower portion of the printer portion 10. The plurality of medium accommodating cassettes 12 are provided at a cassette accommodating portion 14. A medium P accommodated in the medium accommodating cassette 12 passes through a cassette feed path R0, and is sent to a recording transport path R1. Then, recording is performed by a recording section 29 that will be described later. In addition, a feed tray 22 (see FIG. 2) configured to feed the medium one by one through manual feed is provided at a side surface of the printer portion 10. The feed tray 22 rotate with a rotary shaft 22a being the center to switch between an in-use state (not illustrated) and an accommodated state illustrated in FIG. 2. The medium mounted at the feed tray 22 passes through a manual-feeding feed path R6 and is sent to the recording transport path R1, and then, recording is performed by a recording section 29 that will be described later.

Returned to FIG. 1, when double-sided recording is performed, the medium after recording by the recording section 29 is transferred to a switch-back path R2, then, is transferred to an inversion path R3, and is transferred to the recording transport path R1 again. The media having a first surface on which recording has been performed or the medium having both the first surface and a second surface on which recording has been performed are transferred to either one of a face-down ejecting path R4 and a face-up ejecting path R5. The medium that has been transferred to the face-down ejecting path R4 is ejected to an after-recording ejection tray 13 provided above the recording section 29. The medium that has been transferred to the face-up ejecting path R5 is handed over to the intermediate unit 3.

In this manner, the printer portion 10 includes a medium inversion path configured such that, after the first surface of the medium passes through a position that is opposed to the recording section 29, the medium is inverted to cause the second surface that is an opposite surface from the first surface to face the recording section 29. In the present embodiment, this medium inversion path is comprised of the switch-back path R2 and the inversion path R3.

Here, the medium transport path of the printer portion 10 will be further described with reference to FIG. 2. Note that the roller illustrated with the reference character F in FIG. 2 is a roller configured to rotate by receiving power of a motor that is not illustrated, whereas other rollers, to which no reference character is attached, are driven rollers, most of which are spur gears having an outer peripheral surface with gear teeth.

The cassette feed path R0 is a path that extends from the medium accommodating cassette 12 up to a transport roller pair 17A. The recording transport path R1 is a straight-shaped path that extends from the transport roller pair 17A up to a transport roller pair 17B. The switch-back path R2 is a path formed in a curled shape and extending from the transport roller pair 17C to the +Y direction. The inversion path R3 is a curved path that passes from the transport roller pair 17C and above the recording section 29 and reaches the transport roller pair 17A. The face-down ejecting path R4 is a path that extends from the transport roller pair 17B in the +Y direction and curved upward to reach a transport roller pair 17E. The face-up ejecting path R5 is a path that extends from the transport roller pair 17D toward the obliquely downward direction. A manual-feeding feed path R0 is a path that extends from the feed tray 22 up to the transport roller pair 17A.

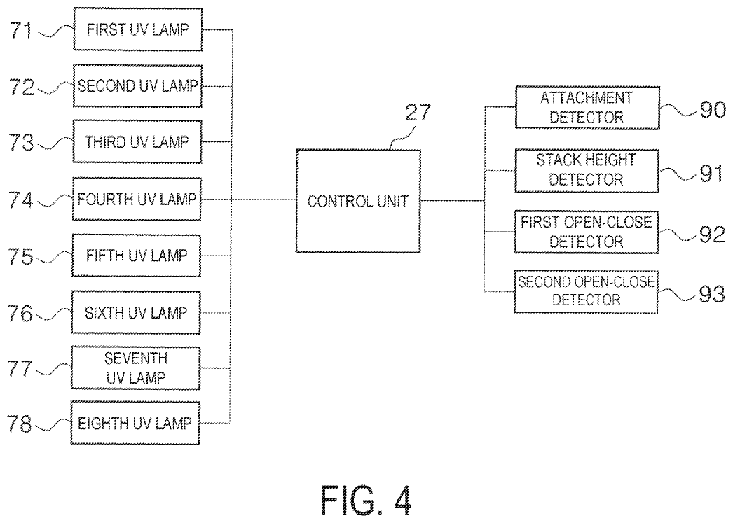

The medium accommodating cassette 12 is provided detachably with respect to a device body 10a of the printer portion 10. The device body 10a includes an attached state detector 90 (see FIG. 4) configured to detect an attached state of the medium accommodating cassette 12. The control unit 27 is able to detect whether or not the medium accommodating cassette 12 is attached, on the basis of a detection signal from the attached state detector 90. In addition, the device body 10a includes a stack height detector 91 (see FIG. 4) configured to detect a stack height of the media in the medium accommodating cassette 12. The control unit 27 is able to detect the stack height of the media in the medium accommodating cassette 12, on the basis of a detection signal from the stack height detector 91. Note that the attached state detector 90 and the stack height detector 91 are each provided at each of a plurality of medium accommodating cassettes 12.

A lift plate 19 is provided in the lower side of the medium accommodating cassette 12 so as to be rotatable with a rotary shaft 19a being the center. As the lift plate 19 rotates with the power of the motor that is not illustrated, the media accommodated in the medium accommodating cassette 12 is pushed up by the lift plate 19, and the uppermost medium is brought into contact with a feed roller 20. The reference character Pa in FIG. 2 represents a medium accommodated in the medium accommodating cassette 12.

The solid line and the reference character 19 in FIG. 2 indicate a lift plate that is in a lowered state, and the long dashed double-short dashed line and the reference character 19-1 indicate a lift plate that is in a raised state. At a feed standby state, the lift plate 19 is in the lowered state. Upon the control unit 27 receiving a recording job, the lift plate 19 switches from the lowered state to the raised state on the basis of control of the control unit 27. Note that, at the time of a recording job indicating that recording is performed on a plurality of media, the lift plate 19 may maintain the raised state, or may be raised and lowered every time one medium is fed.

The feed roller 20 receives the power of the motor, which is not illustrated, to rotate, and sends out the uppermost medium from the medium accommodating cassette 12. The feed roller 20 constitutes a feeding portion configured to send out the medium from the medium accommodating cassette 12. The medium that has been sent out receives a separation effect from a separating roller pair 21 to be separated, and reaches a transport roller pair 17F. Then, the medium is transported by the transport roller pair 17F to the transport roller pair 17A.

The feed tray 22 is configured to rotate with the rotary shaft 22a being the center, thereby being able to switch between the accommodated state illustrated in FIG. 2 and an expanded state that is not illustrated. In the expanded state, the feed tray 22 supports a medium in a tilted posture. The medium supported by the feed tray 22 is transported to the transport roller pair 17A by the feed roller 15. Note that the reference character 16 represents a separating roller configured to nip a medium between the separating roller and the feed roller 15 to separate it.

Next, in the recording transport path R1, the recording sheet receives transporting force from the transport roller pair 17A and a belt unit 18, and is transported in the +Y direction. The belt unit 18 includes a transport belt 18c. The transport belt 18c is an endless belt made out of a base material made of urethane, rubber, or the like in which a conductive material is included as necessary to adjust a resistance value. The transport belt 18c is looped around a driving pulley 18a at the upstream side and a driven pulley 18b at the downstream side. A predetermined tension is applied to the transport belt 18c by a tensioner (not illustrated).

The driving pulley 18a is rotationally driven by a motor (not illustrated). When the driving pulley 18a is rotationally driven, the transport belt 18c circles around the driving pulley 18a and the driven pulley 18b. This causes the recording sheet adsorbed to the transport belt 18c to be transported.

A charge roller (not illustrated) is in contact with the transport belt 18c and is rotationally driven in response to circling operation of the transport belt 18c. A power supply device (not illustrated) configured to apply DC voltage to the charge roller is coupled to the charge roller and the charge roller supplies an electrical charge to a site that is in contact with the transport belt 18c, and thus the recording sheet is electrostatically adsorbed to the transport belt 18c.

The recording section 29 configured to perform recording on a medium is provided at a position that is opposed to the belt unit 18. The recording section 29 includes a liquid discharging head 24 configured to discharge ink serving as one example of a liquid. The liquid discharging head 24 according to the present embodiment is a liquid discharging head configured such that a plurality of ink discharging nozzles (not illustrated) are provided so as to cover the entire area of the X-axis direction, that is, of the medium width direction, and is configured as a liquid discharging head that can perform recording on the entire width of a medium without moving in the X-axis direction, that is, the medium width direction. Note that ink is supplied to the liquid discharging head 24 through an ink tube, which is not illustrated, from a liquid accommodating portion, which is not illustrated.

A flap 25A is provided downstream of the belt unit 18. This flap 25A allows the transport direction of the medium to be switched into either the obliquely upward direction or the straight direction. When the transport direction of the medium is directed into the obliquely upward direction by the flap 25A, the medium enters the switch-back path R2. A flap 25B is provided in front of the entrance to the switch-back path R2. When the medium travels from the switch-back path R2 toward the inversion path R3, the medium is guided to the inversion path R3 by the flap 25B.

In addition, a flap 25C is provided near the transport roller pair 17D. This flap 25C allows the transport destination of the medium to be switched into either the face-down ejecting path R4 or the face-up ejecting path R5.

The medium ejected from the face-down ejecting path R4 is ejected by the transport roller pair 17E toward the after-recording ejection tray 13. The reference character Pb represents a medium supported by the after-recording ejection tray 13.

Note that the reference character 83 represents a first cover. This first cover 83 is provided so as to be attachable to and detachable from the front face of the device body 10a. In the attached state, the first cover 83 covers a space above the after-recording ejection tray 13, that is, a medium ejection space, and the medium Pb cannot be seen from the outside. By detaching the first cover 83 from the device body 10a, it is possible to remove the medium Pb supported by the after-recording ejection tray 13.

In addition, a second cover 84 is provided at the front face of the device body 10a in a detachable manner. Once the second cover 84 is detached, part of the recording transport path R1, part of the switch-back path R2, part of the inversion path R3, and part of the face-down ejecting path R4 are exposed. Thus, by detaching the second cover 84, it is possible to access the medium transport path inside of the device to remove a medium when a jam occurs.

Note that the device 10a includes a first open-close detector 92 (see FIG. 4) configured to detect an open-close state of the first cover 83. The control unit 27 is able to detect whether the first cover 83 is opened or closed, on the basis of a detection signal from the first open-close detector 92. Similarly, the device body 10a includes a second open-close detector 93 (see FIG. 4) configured to detect whether the second cover 84 is opened or closed. The control unit 27 is able to detect whether the second cover 84 is opened or closed, on the basis of a detection signal from the second open-close detector 93.

Next, returned to FIG. 1, the intermediate unit 3 will be described. The intermediate unit 3 hands the medium received from the recording unit 2 over to the post-processing unit 5. The intermediate unit 3 is disposed between the recording unit 2 and the post-processing unit 5. The medium transported through the face-up ejecting path R5 of the recording unit 2 is received by the intermediate unit 3 through a reception path 30, and is transported toward the post-processing unit 5.

The intermediate unit 3 includes two transport paths configured to transport the medium. The first transport path is a path configured to extend from the reception path 30 through a first switch-back path 31 indicated as the dashed line in FIG. 1 and then transport the medium to a merging path 33. The second path is a path configured to extend from the reception path 30 through a second switch-back path 32 indicated as the long dashed double-short dashed line in FIG. 1 and then transport the medium to the merging path 33.

The first switch-back path 31 is a path configured to receive the medium in a direction of the arrow A1, and then perform switch-back of the medium toward a direction of the arrow A2. The second switch-back path 32 is a path configured to receive the medium in a direction of the arrow B1, and then perform switch-back of the medium toward a direction of the arrow B2.

The reception path 30 branches off, at a branch portion 35, into the first switch-back path 31 and the second switch-back path 32. A flap, not illustrated, configured to switch the transport destination of the medium into either the first switch-back path 31 or the second switch-back path 32 is provided at the branch portion 35.

Furthermore, the first switch-back path 31 and the second switch-back path 32 merge at a merging unit 36. Thus, regardless of whether the medium is sent from the reception path 30 to the first switch-back path 31 or the second switch-back path 32, it is possible to hand the medium over to the post-processing unit 5 via the common merging path 33.

The intermediate unit 3 receives the medium from the recording unit 2 into the reception path 30 with the most recent recording surface facing up. However, the medium is curved and inverted at the merging path 33 to be the most recent recording surface facing down.

Thus, the medium with the most recent recording surface facing down is handed over from the +Y direction of the intermediate unit 3 to a first transport path 43 of the post-processing unit 5.

Note that the receiving path 30, the first switchback path 31, the second switchback path 32, and the merged path 33 are each provided with one or more roller pairs (not illustrated) as one example of a unit that transports a medium.

When recording is performed continuously on a plurality of media in the recording unit 2, the media that enter the intermediate unit 3 are alternately fed to a transport path passing through the first switch-back path 31 and a transport path passing through the second switch-back path 32. This makes it possible to enhance the throughput of medium transport in the intermediate unit 3.

Further, in a case of the configuration in which ink is discharged to a medium to perform recording as in the recording unit 2 according to the present embodiment, when the medium is wet at the time of performing processing in the post-processing unit 5 and a second unit 6 in the subsequent stage, the recording surface may be rubbed off and the alignment of the medium may be poor.

By handing over the medium after recording from the recording unit 2 to the post-processing unit 5 through the intermediate unit 3, it is possible to prolong the transport period until the medium after recording is transported to the post-processing unit 5, and also possible to further dry the medium before it reaches the post-processing unit 5 or the second unit 6.

Next, the post-processing unit 5 will be described. The post-processing unit 5 illustrated in FIG. 1 includes a reception portion 41 provided in a lower portion of the −Y direction and configured to receive a medium from the intermediate unit 3. The medium transported through the merging path 33 of the intermediate unit 3 enters the interior of the post-processing unit 5 from the reception portion 41, and is handed over to the first transport path 43.

The post-processing unit 5 includes a drying portion 50 configured to perform a drying process to the medium received from the reception portion 41, and an end binding portion 42 configured to perform a process to the medium received from the reception portion 41 or a medium processed by the drying portion 50.

The post-processing unit 5 includes the first transport path 43 configured to transport the medium received from the reception portion 41, and a second transport path 44 configured to branch off from the first transport path 43 at a first branch portion D1 and send the medium to the drying portion 50. A flap, not illustrated, configured to switch the transport destination of the medium between the first transport path 43 and the second transport path 44 is provided at the first branch portion D1.

The end binding portion 42 is a component configured to perform an end binding process used to bind end portions of media, such as a corner portion on one side of a medium or a side of a medium at one side. The end binding portion 42 is configured to include a stapler as one example.

The drying portion 50 is a component configured to apply a drying process to a medium. In the present embodiment, the drying portion 50 heats a medium to dry the medium.

More specifically, the drying portion 50 includes a heat roller pair 51 serving as a dry processing unit configured to perform a drying process to a medium, and a loop-shape transport path 52 including the heat roller pair 51 and configured to be able to transport the medium in a circling manner. The second transport path 44 branched off from the first transport path 43 merges, at a position upstream of the heat roller pair 51, with the loop-shape transport path 52, and transports the medium using a transport roller pair 63 provided at the second transport path 44, thereby being able to introduce it into the loop-shape transport path 52.

In the present embodiment, the heat roller pair 51 includes a lower roller serving as a dry driving roller that is driven by a drive source (not illustrated) and also includes an upper roller serving as a dry driven roller that is rotationally driven by the rotation of the dry driving roller. The dry driving roller is heated by a heater (not illustrated). Thus, heat builds up at the dry driving roller to dry the medium.

Note that, in a state where the most recent recording surface faces downward, the medium transported from the intermediate unit 3 passes from the reception portion 41 of the post-processing unit 5 through the first transport path 43, and enters the second transport path 44. The medium is then nipped by the heat roller pair 51 with the most recent recording surface facing downward. Thus, in the heat roller pair 51, a roller to be heated is a roller configured to be brought into contact with the most recent recording surface of the medium.

The drying portion includes the loop-shape transport path 52 and is configured to be able to transport the medium in a circling manner at the loop-shape transport path 52. Thus, by transporting a medium in a circling manner a plurality of times, it is possible to perform the drying process using the heat roller pair 51 a plurality of times. This makes it possible to further reliably dry the media.

The medium after drying by the drying portion 50 is transported to a fourth transport path 59 coupled to the loop-shape transport path 52 and merges into the first transport path 43 at a second merging unit G2.

In addition, the post-processing unit 5 includes the hole-creating mechanism portion 46 configured to perform a hole creating process to a medium received from the reception portion 41. The hole-creating mechanism portion 46 is provided at a position near the reception portion 41 on the first transport path 43 through which the medium received by post-processing unit 5 passes. In addition, the hole-creating mechanism portion 46 is configured to be able to perform the hole creating process upstream of the first transport path 43.

The medium received from the reception portion 41 can be transported to a process tray 48. At the process tray 48, the media are stacked at the process tray 48 with a trailing edge in the transport direction being aligned. When a predetermined number of media is stacked at the process tray 48, the end binding process by the end binding portion 42 can be performed to the trailing edges of the media.

The medium processed by the end binding portion 42 is ejected by an ejecting unit, not illustrated, from a first ejecting portion 60 to the outside of the device of the post-processing unit 5, and then, is placed at a first tray 40.

In addition, at a second branch portion D2 disposed downstream of the first branch portion D1, the first transport path 43 is coupled to a third transport path 45 branched off from the first transport path 43. A flap, not illustrated, configured to switch the transport destination of the medium between the first transport path 43 and the third transport path 45 is provided at the second branch portion D2.

An upper tray 49 is provided in an upper portion of the post-processing unit 5. The third transport path 45 continuously extends from the second branch portion D2 to a second ejecting portion 61, and the medium transported through the third transport path 45 is ejected from the second ejecting portion 61 to the upper tray 49. In other words, the medium received from the reception portion 41 can be ejected at the upper tray 49 without passing through the end binding portion 42.

Next, ultraviolet-light emission to a medium in the recording unit 2 will be described. The recording unit 2 includes a UV lamp serving as an ultraviolet-light emitting portion configured to emit ultraviolet light to a medium. The recording unit 2 includes UV lamps provided at a plurality of locations, and these lamps are indicated in FIG. 2 as reference characters 71, 72, 73, 74, 75, 76, 77, and 78. The reference character 71 represents a first UV lamp. Similarly, the reference character 72 represents a second UV lamp. The reference character 73 represents a third UV lamp. The reference character 74 represents a fourth UV lamp. The reference character 75 represents a fifth UV lamp. The reference character 76 represents a sixth UV lamp. The reference character 77 represents a seventh UV lamp. The reference character 78 represents an eighth UV lamp. Turning on and off of each of the UV lamps are controlled by the control unit 27 as shown in FIG. 4.

The wavelength of ultraviolet light emitted by each of the UV lamps falls in a range of 200 to 280 nm. In particular, the present embodiment employs UV lamps of which peak wavelength is 260 nm. By emitting ultraviolet light to a medium by using such UV lamps, a sterilization process is applied to bacteria or virus attached on the medium.

Note that the sterilization as used herein means reducing at least either bacteria or virus.

All the UV lamps illustrated in FIG. 2 are UV lamps that extend in the X-axis direction so as to be able to irradiate the entire area of a medium in the +X direction.

The first UV lamp 71 is provided at a position that is opposed to the medium accommodating cassette 12, and is configured to irradiate the entire upper surface of the uppermost medium of the media accommodated in the medium accommodating cassette 12. Note that the end portion of the first UV lamp 21 in the −Y direction is not opposed to the end portion, in the −Y direction, of a medium accommodated in the medium accommodating cassette 12. However, with ultraviolet light emitted in a manner illustrated by the arrow c, the end portion, in the −Y direction, of the medium accommodated in the medium accommodating cassette 12 is also irradiated with the ultraviolet light. Note that, hereinafter, the upper surface of a medium accommodated in the medium accommodating cassette 12 is referred to as a “second surface,” and the lower surface of the medium accommodated in the medium accommodating cassette 12 is referred to as a “first surface.” When recording is performed on both sides of a medium fed from the medium accommodating cassette 12, recording is first performed on the first surface, and then, recording is performed on the second surface. For a medium set at the feed tray 22, the upper surface on which recording is first performed is referred to as a first surface, and the lower surface on which recording is performed next is referred to as a second surface.

The second UV lamp 72 and the third UV lamp 73 are provided in the cassette feed path R0. The second UV lamp 72 is configured to emit ultraviolet light to the second surface of the medium sent out from the medium accommodating cassette 12. The third UV lamp 73 is configured to emit ultraviolet light to the first surface of the medium sent out from the medium accommodating cassette 12.

The fourth UV lamp 74 and the fifth UV lamp 75 are provided in the recording transport path R1. The fourth UV lamp 74 is configured to emit ultraviolet light to the second surface of a medium. The fifth UV lamp 75 is configured to emit ultraviolet light to the first surface of the medium. Note that the fourth UV lamp 74 and the fifth UV lamp 75 are each able to emit ultraviolet light to a medium that has been inverted to perform recording on both sides. When the medium is inverted for the purpose of both side recording, the fourth UV light 74 is able to emit ultraviolet light to the first surface of the medium, that is, to a surface on which recording has been already performed. In addition, the fifth UV lamp 75 is able to emit ultraviolet light to the second surface of the medium, that is, to a surface on which recording has not yet been performed.

The sixth UV lamp 76 and the seventh UV lamp 77 are provided near the transport roller pair 17C that is an entrance to the switch-back path R2. The sixth UV lamp 76 is configured to emit ultraviolet light to the second surface of a medium. The seventh UV lamp 77 is configured to emit ultraviolet light to the first surface of the medium.

The eighth UV lamp 78 is provided at a position that is opposed to a record-section ejecting tray 13, and is configured to irradiate the entire upper surface of a medium ejected to the record-section ejecting tray 13. Note that, depending on details of recording, the upper surface of a medium ejected to the record-section ejecting tray 13 may be the first surface or may be the second surface.

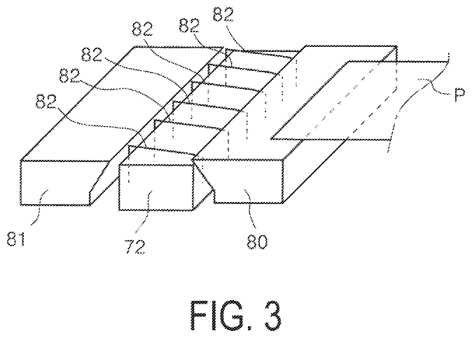

The UV lamps other than the first UV lamp 71 and the eighth UV lamp 78 are provided in the medium transport path, and emit ultraviolet light to a transported medium to irradiate the entire surface of the first surface or the second surface of the medium with the ultraviolet light. It is preferable that the UV lamps provided in the medium transport path are each provided with a wire as illustrated in FIG. 3. In FIG. 3, wires 82 that the second UV lamp 72 includes are illustrated as one example. The reference character 80 represents a path formation member disposed upstream of the second UV lamp 72 in the medium transport path. The reference character 81 represents a path formation member disposed downstream of the second UV lamp 72 in the medium transport path. The wire 82 guides the medium P so as to advance from the path formation member 80 to the path formation member 81 so that the medium P does not get caught. By providing the wire 82 at a position of the UV lamp in this manner, it is possible to suppress a reduction in the intensity of the ultraviolet light, as compared with a case where the medium P is guided by glass or other members that allow ultraviolet light to pass through.

Note that it is preferable that the wire 82 is disposed at a sloped angle relative to the medium transport direction. This makes it possible to prevent the wire 82 from forming a shadow when ultraviolet light is emitted to the medium P.

Not all the plurality of UV lamps described above need to be provided. It is only necessary that only the necessary UV lamps are provided in accordance with individual examples of controlling of emission of ultraviolet light described below. However, it is needless to say that all the UV lamps described above may be provided.

In addition, the examples of controlling of emission of ultraviolet light described below may be combined and be used as long as it is not technically contradict.

First Control Example

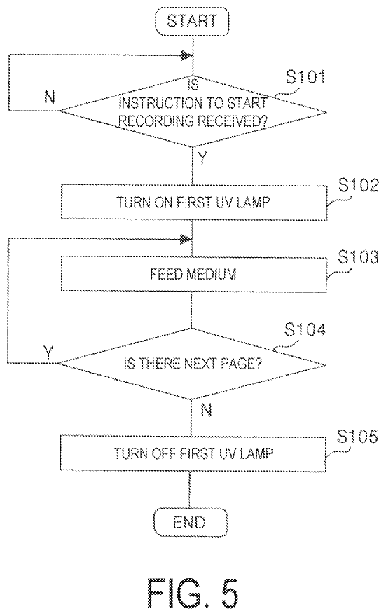

A first control example concerning emission of ultraviolet light to a medium will be described with reference to FIG. 5. The present control example employs the first UV lamp 71.

Upon receiving an instruction to start recording (Yes in step S101), the control unit 27 turns on the first UV lamp 71 (step S102). After this, the control unit 27 feeds a medium (step S103). Once feeding of a medium on the preceding page is completed, the control unit 27 feeds a medium on the next page when the next page exists (Yes in step S104). When the next page does not exist (No in step S104), the control unit 27 turns off the first UV lamp 71 (step S105).

By using the first UV lamp 71 configured to emit ultraviolet light toward a medium accommodated in the medium accommodating cassette 12 in this manner, it is possible to emit the ultraviolet light to the medium by utilizing a period of time before the medium starts to be fed. This makes it possible to prevent the recording throughput from reducing in association with ultraviolet-light irradiation. In the control example shown in FIG. 4, the entire second surface of a medium on the first page is irradiated with ultraviolet light in a state where the medium is at rest and during a period of time from the start of turning on the first UV lamp 71 to the completion of raising the lift plate 19. Note that, even after the medium starts to be fed, the ultraviolet light is emitted to a region of the second surface that is opposed to the first UV lamp 71.

Media on and after the second page are irradiated with ultraviolet light such that, as a medium on the preceding page disappears from above a medium on the following page, ultraviolet light is emitted to the following page. Thus, in the recording job in which recording is performed on a plurality of media, it is more preferable that the lift plate 19 descends every time feeding of one sheet of medium is completed, from the viewpoint of ultraviolet-light irradiation. This makes it possible to avoid occurrence of a region where the feed roller 20 makes a shadow, so that ultraviolet light is not emitted to a medium.

In addition, it is preferable set a predetermined standby period before step S103 in FIG. 4 is implemented.

In addition, as illustrated in FIG. 5, at the time of feeding a plurality of media, the control unit 27 sets the first UV lamp 71 in an emitting state before the first medium starts to be fed, and then, maintains the emitting state at least until feeding of the last medium in the recording job is completed. This makes it possible to prevent the recording throughput from reducing in association with ultraviolet-light irradiation while securing a period time for emission of ultraviolet light to the media. Note that, for example, the first UV lamp 71 can be turned off at the timing when a medium detecting sensor (not illustrated) provided near the upstream of the transport roller pair 17F detects that the trailing end of the last medium has passed.

Note that the control unit 27 may bring the first UV lamp 71 into the emitting state when the power supply for the device is turned on. This makes it possible to immediately start to feed the media upon receiving an instruction to start recording, which makes it possible to further prevent the recording throughput from reducing in association with ultraviolet-light irradiation. Note that turning of the power supply for the device may include returning from a power save mode.

Furthermore, it is preferable that the present control example further employs the eighth UV lamp 78, in addition to the first UV lamp 71. The eighth UV lamp 78 is provided at a position that is opposed to the record-section ejecting tray 13, and is configured to irradiate the entire upper surface of a medium ejected to the record-section ejecting tray 13. This makes it possible to suppress a reduction in the recording throughput at the time of emitting ultraviolet light to the medium.

Incidentally, in the first control example described above, the sterilization process is performed only to the second surface of a medium accommodated in the medium accommodating cassette 12. Thus, in a case of the present control example alone, the sterilization process is not performed to the first surface. However, the sterilization process can be performed to the first surface by using other UV lamps. For example, by sending a medium on which recording has been performed, to the switch-back path R2, the sterilization process may be performed by using the seventh UV lamp 77, and also may be performed by using the eighth UV lamp 78 in the manner described above.

Furthermore, the sterilization process to the first surface of a medium may be omitted on an as-necessary basis. For example, at the time of setting a medium at the medium accommodating cassette 12, a user touches only the front surface and the back surface of a bundle of media, in most cases. Thus, by performing the sterilization process only to the front surface and the back surface of a bundle of media, it is possible to efficiently obtain the sterilization effect.

Specifically, when the first recording job is received from a time when the medium accommodating cassette 12 is attached, the sterilization process is performed to the front surface of the bundle of media by performing the sterilization process to the first medium on the basis of the first control example described above.

Next, the sterilization process for the back surface of a bundle of media can be performed in the following manner. For example, the height of media loaded within the cassette is acquired on the basis of a detection signal from a loading height detector 91 (see FIG. 4), and then, the range of the number of media that exist above the lowermost medium is obtained on the basis of the estimated minimum value of and the estimated maximum value of the medium thickness. For example, when it is estimated that the number of sheets fed after attachment of the medium accommodating cassette 12 falls in a range of 99 sheets to 119 sheets, and this range of sheets had existed above the lower most medium, it may be possible to employ a configuration in which the sterilization process to the first surface is performed for the media that fall in this range in addition to the sterilization process to the second surface, and the sterilization process to the first surface is omitted for the second to 99th media.

Note that, when additional media are added in a state where a medium remains in the medium accommodating cassette 12, the range of the number of media that exist above the lowermost medium is sequentially obtained again.

Second Control Example

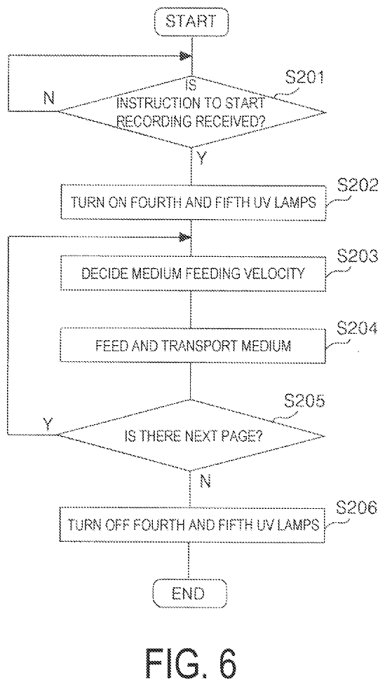

A second control example concerning emission of ultraviolet light to a medium will be described with reference to FIG. 6. The present control example employs the fourth UV lamp 74 and the fifth UV lamp 75.

Upon receiving an instruction to start recording (Yes in step S201), the control unit 27 turns on the fourth UV lamp 74 and the fifth UV lamp 75 (step S202). Then, the control unit 27 decides a medium feeding velocity (step S203). Note that the medium feeding velocity as used herein means a velocity of a medium at which the medium passes through a region that is opposed to the fourth UV lamp 74 and a region that is opposed to the fifth UV lamp 75. By increasing and decreasing this velocity, the period of time for emitting ultraviolet light to a medium is controlled. The velocity of a medium at which the medium passes through the region that is opposed to the fourth UV lamp 74 and the region that is opposed to the fifth UV lamp 75 is controlled by controlling a motor (not illustrated) serving as a drive source for a roller used to feed and transport the medium.

Next, the control unit 27 causes a medium to be fed and transported on the basis of the decided medium feeding velocity (step S204). After this, once recording on the preceding page is completed, the control unit 27 causes a medium on the next page to be fed and transported when this next page exists (Yes in step S205). When the next page does not exist (No in step S205), the control unit 27 turns off the fourth UV lamp 74 and the fifth UV lamp 75 (step S206).

Here, decision of the medium feeding velocity in step S203, in other words, decision of the period of time for emitting ultraviolet light to a medium is made on the basis of a period of time that has elapsed since the medium accommodating cassette 12 is brought into the attached state. In other words, bacteria or virus attached on the media die over time, and the entire number thereof reduces, accordingly. Thus, the period of time for emitting ultraviolet light can be set to be short. On the basis of such a property, the present control example is configured such that the control unit 27 sets the period of time for emitting ultraviolet light to be shorter when an elapsed period from a time when the medium accommodation portion 12 changes into the attached state is a first period, than when the elapsed period is a second period that is shorter than the first period. This makes it possible to further prevent the recording throughput from reducing in association with ultraviolet-light irradiation.

Thus, in a case of employing the present control example, when the medium accommodating cassette 12 changes into the attached state, the control unit 27 writes data and time information about this into a non-volatile memory that is not illustrated. Then, at the time of deciding the medium feeding velocity (step S203), the data and time information is read out. The medium feeding velocity may be decided by storing, in a non-volatile memory, a setting table that defines a relationship between the period of time elapsed from a time when the medium accommodating cassette 12 changes into the attached state and the medium feeding velocity, and reading it out, or may be obtained for every occasion using a computational expression.

Note that, at the time of deciding the medium feeding velocity in step S203, it may be possible to use detection information from the stack height detector 91 (see FIG. 4) used to detect the stack height of media within the medium accommodating cassette 12, instead of the information about a period of time elapsed from a time when the medium accommodating cassette 12 changes into the attached state described above. As described above, bacteria or virus attached on the media die over time, and the entire number thereof reduces, accordingly. In response to this, the period of time for emitting ultraviolet light can be set to be short. Thus, it can be said that the period of time for emitting ultraviolet light can be reduced with decrease in the stack height of media within the medium accommodating cassette 12. Thus, when the stack height is the first height, the control unit 27 may set the period of time for emitting ultraviolet light to be lower than when the stack height is the second height that is lower than the first height. In other words, the control unit 27 reduces the period of time for emitting ultraviolet light with decrease in the number of media within the medium accommodating cassette 12. This make it possible to further prevent the recording throughput from reducing in association with ultraviolet-light irradiation.

Note that it is preferable to apply the second control example described above in combination with the first control example described above, in addition to using the second control example alone. In this case, the first UV lamp 71 serves as one example of a first ultraviolet-light emitting portion, and the fourth UV lamp 74 and the fifth UV lamp 75 serve as one example of a second ultraviolet-light emitting portion.

In addition, in a case where the second control example is applied in combination with the first control example, when ultraviolet light is emitted toward a medium fed from the feed tray 22, it is preferable to use an equal period as the period of time for emitting the ultraviolet light. This is because, in the present embodiment, the feed tray 22 is a feed tray in which media are set one by one and are fed, and thus, the period elapsed from a time when a user touches the media set at the feed tray 22 is short, which makes it preferable to secure the period of time for emitting ultraviolet light.

Third Control Example

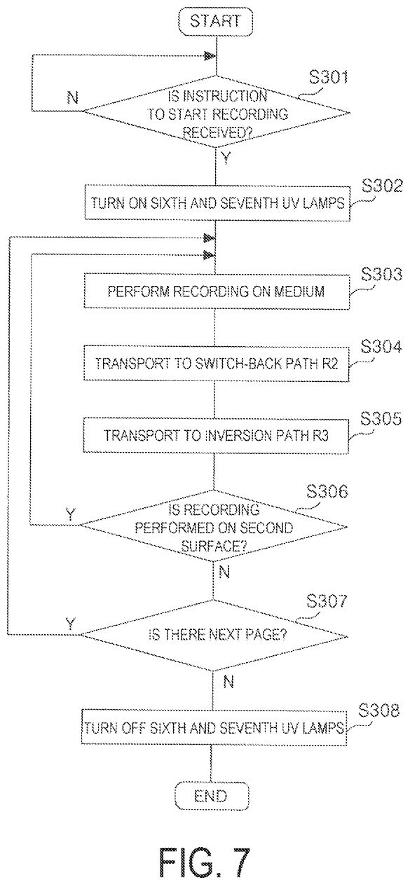

A third control example concerning emission of ultraviolet light to a medium will be described with reference to FIG. 7. The present control example employs the sixth UV lamp 76 and the seventh UV lamp 77. Upon receiving an instruction to start recording (Yes in step S301), the control unit 27 turns on the sixth UV lamp 76 and the seventh UV lamp 77 (step S302). Then, the control unit 27 causes recording to be performed on the first surface of a medium (step S303). Then, even when recording is performed only on the first surface, the medium is transported to the switch-back path R2 (step S304). After this, the medium is transported to the inversion path R3 (step S305). Here, when recording is also performed on the second surface of the medium (Yes in step S306), the process is performed again from step S303. Note that, when recording is performed on the first surface and then recording is also performed on the second surface, determination in step S306 results in “No,” and the medium is ejected. This procedure is performed until the last medium in the recording job. Once recording on the last medium in the recording job is completed (No in step S307), the sixth UV lamp 76 and the seventh UV lamp 77 are turned off (step S308).

In the printer portion 10, the switch-back path R2 including the sixth UV lamp 76 and the seventh UV lamp 77 can be used as a first transport region configured to reduce the medium transport velocity to transport media. By using the switch-back path R2 as the first transport region as described above, it is possible to promote drying of the first surface of the medium to which ink has been discharged. Note that “reducing the medium transport velocity” means reducing the medium transport velocity relative to a medium transport velocity before the medium is sent to the switch-back path R2. Note that, in the present example, after the medium enters the switch-back path R2, the transport direction of the medium is switched. Then, the medium transport velocity at the time of transporting it to the inversion path R3 is reduced to create a dry waiting period.

At this time, the medium is irradiated with ultraviolet light from the sixth UV lamp 76 and the seventh UV lamp 77. Thus, by using the dry waiting period, it is possible to emit ultraviolet light, which makes it possible to prevent the recording throughput from reducing in association with ultraviolet-light irradiation.

Note that the dry waiting period can be created by reducing the transport velocity of the media as described above. This reduction in the transport velocity includes setting the transport velocity to zero, that is, includes stopping transporting the media. Thus, for example, it may be possible to repeat a procedure in which, after ultraviolet light is emitted to a predetermined region of the medium in a state where transporting of the medium is stopped, the medium is transported to emit ultraviolet light to the next predetermined region of the medium; and then, ultraviolet light is emitted to the predetermined region of the medium in a state where transporting of the medium is stopped again.

Note that, at the time of switching the transport direction of the medium after the medium enters the switch-back path R2, the end portion of the medium at the −Y direction is nipped by the transport roller pair 17C. This leads to a possibility that ultraviolet light is not sufficiently emitted to a portion of the medium that is nipped by the transport roller pair 17C. In this case, it may be possible to use the fourth UV lamp 74 and the fifth UV lamp 75 disposed upstream of the liquid discharging head 24 to emit ultraviolet light only to the above-described region that is not sufficiently irradiated with ultraviolet light.

Furthermore, in the present example, the sixth UV lamp 76 and the seventh UV lamp 77 are provided near the +Y direction of the transport roller pair 17C that is an entrance to the switch-back path R2. Thus, it is possible to emit ultraviolet light to a medium by using both the time when the medium enters the switch-back path R2 and the time when the medium exits the switch-back path R2. This makes it possible to prevent the recording throughput from reducing in association with ultraviolet-light irradiation while securing the period of time for emitting ultraviolet light. Note that the sixth UV lamp 76 and the seventh UV lamp 77 may be provided near the −Y direction of the transport roller pair 17C, instead of being provided near the +Y direction of the transport roller pair 17C or in addition to being provided near the +Y direction of the transport roller pair 17C.

Note that, in addition to using the third control example alone, it is also preferable to use the third control example described above in combination with the first control example or the second control example described above. When the first UV lamp 71 is used in a combined manner, the first UV lamp 71 serves as one example of a first ultraviolet-light emitting portion, and the sixth UV lamp 76 and the seventh UV lamp 77 serve as one example of a second ultraviolet-light emitting portion.

Fourth Control Example

A third control example concerning emission of ultraviolet light to a medium will be described. The present control example employs the fourth UV lamp 74 and the fifth UV lamp 75. Upon receiving an instruction to start recording, the control unit 27 turns on the fourth UV lamp 74 and the fifth UV lamp 75. The state of the fourth UV lamp 74 and the fifth UV lamp 75 being turned on is kept until the last medium in the recording job is ejected.

In the present example, the liquid discharging head 24 is configured so as to also move in the X-axis direction, that is, in the medium width direction. In other words, the printer portion 10 is configured as a serial-type recording device.

In a case where the printer portion 10 is configured as a serial-type recording device, recording is performed on a medium by alternately performing an operation of moving the liquid discharging head 24 in the X-axis direction and operation of transporting the medium by a predetermined amount. In addition, when the operation of moving the liquid discharging head 24 in the X-axis direction is performed, the medium is temporarily stopped. By using this period of time, the fourth UV lamp 74 and the fifth UV lamp 75 are used to emit ultraviolet light to the medium. This makes it possible to prevent the recording throughput from reducing in association with ultraviolet-light irradiation. In other words, the control unit 27 overlaps, with each other in terms of time, the operation of discharging ink using the liquid discharging head 24 and emission of ultraviolet light using the fourth UV lamp 74 and the fifth UV lamp 75. This makes it possible to prevent the recording throughput from reducing in association with ultraviolet-light irradiation.

Note that, in addition to using the fourth control example alone, it is also preferable to use the fourth control example in combination with the first control example, the second control example, and the third control example described above. When the first UV lamp 71 is used in a combined manner, the first UV lamp 71 serves as one example of a first ultraviolet-light emitting portion, and the fourth UV lamp 74 and the fifth UV lamp 75 serve as one example a second ultraviolet-light emitting portion.

Fifth Control Example

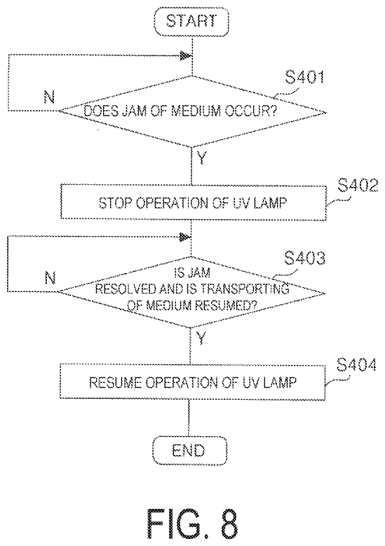

A fifth control example concerning emission of ultraviolet light to a medium will be described with reference to FIG. 8. In the present example, at least any one of the first to fourth control examples described above is implemented, and at least any one of the plurality of UV lamps emits ultraviolet light to the medium.

The printer portion 10 includes a medium detecting sensor (not illustrated) disposed at an appropriate location in the medium transport path and configured to detect passage of a medium. This medium detecting sensor can be provided at an appropriate location such as the vicinity of a location disposed upstream of the transport roller pair 17A, between the transport roller pair 17A and the belt unit 18, and between the transport roller pair 17B and the transport roller pair 17C. When no medium is detected by the medium detecting sensor even if the medium is transported by a predetermined amount, the control unit 27 determines that a jam occurs, on the basis of detection information from the medium detecting sensor.

In FIG. 8, upon detecting that a jam of a medium occurs (Yes in step S401), the control unit 27 stops operation of a UV lamp (step S402). Stopping the operation of a UV lamp includes a case where a UV lamp in operation is turned off, and also includes a case where a UV lamp planned to be turned on is not turned on. Furthermore, upon determining that the jam is resolved and transporting the medium can be resumed (Yes in step S403), the control unit 27 resumes turning on the UV lamp (step S404). For example, determination in step S403 is made such that, when the opened state of the first cover 83 is detected, the closed state of the first cover 83 is detected, and then, a user operates the operation unit 28 to perform an input operation indicating that a jam process is performed, it is determined that the jam is resolved and transporting of the medium can be resumed.

In this manner, when a jam occurs in the medium transport path, the control unit 27 stops emission of ultraviolet light to a medium. This makes it possible to prevent the ultraviolet light from exerting an adverse effect on a user when the user opens the first cover 83 for the purposed of handling of the jam.

Note that, in place of the fifth control example or in addition to the fifth control example, control may be performed so as to stop emission of ultraviolet light to a medium at the time of detecting that the first cover 83 switches from the closed state to the opened state on the basis of a detection signal from the first open-close detector 92 (see FIG. 4). This makes it possible to prevent the ultraviolet light from exerting an adverse effect on a user when the user opens the first cover 83 to access the interior of the device, regardless whether or not occurrence of a jam exists.

Alternatively, it may be possible to employ a configuration in which a lock unit configured to lock the closed state of the first cover 83 is provided, and the control unit 27 performs control such that the first cover 83 is brought into the locked state by using the lock unit during emission of ultraviolet light.

In addition, it is preferable to perform control so as to stop operation of the first UV lamp 71 when the medium accommodating cassette 12 is detached from the device body 10a, on the basis of a detection signal from the attached state detector 90 (see FIG. 4).

Furthermore, it is also preferable to perform control so as to stop operation of the eighth UV lamp 78 at the time of detecting that the second cover 84 switches from the closed state to the opened state, on the basis of a detection signal from the second open-close detector 93 (see FIG. 4).

Note that the present disclosure is not intended to be limited to the aforementioned embodiments, and many variations are possible within the scope of the present disclosure as described in the appended claims. It goes without saying that such variations also fall within the scope of the present disclosure.

For example, in the Examples described above, recording on media and the sterilization process using the UV lamps are performed. However, it may be possible to provide a mode in which only the sterilization process using the UV lamps is performed without performing the recording.

Furthermore, when ultraviolet light is emitted to a medium using the UV lamp, it may be possible to employ a configuration in which air outside of the device is taken into the interior of the device; the air taken in passes through a region where ultraviolet light is emitted; and the air is ejected to the outside by using an extraction fan. This makes it possible to perform sterilization to the air outside of the device and to eject, to the outside of the device, heat generated from the UV lamp.

In addition, it may be possible to further provide a configuration in which air is jetted to a medium; the air is collected; and ultraviolet light is emitted to the air.

What is claimed is:

1. A recording device comprising:
a medium accommodation portion configured to accommodate a plurality of media;
a feeding portion configured to send out the media from the medium accommodation portion;
a recording section configured to perform recording on the media sent out from the medium accommodation portion;
an ultraviolet-light emitting portion provided at a position opposed to the medium accommodation portion and configured to emit ultraviolet light toward the media accommodated in the medium accommodation portion; and
a control unit configured to control the ultraviolet-light emitting portion to emit ultraviolet light to the media by utilizing a period before the feeding portion starts to feed the media.

2. The recording device according to claim 1, wherein,
when feeding the plurality of media, the control unit sets the ultraviolet-light emitting portion in an emitting state before starting to feed a first one of the media, and then, keeps the emitting state at least until completion of feeding of a last one of the media.

3. The recording device according to claim 1, wherein
the control unit sets the ultraviolet-light emitting portion in an emitting state when a power supply for the device is turned on.

4. The recording device according to claim 1, wherein
the medium accommodation portion is attachable to and detachable from a device body including the recording section,
the device body includes:
an attached state detector configured to detect an attached state of the medium accommodation portion; and
a second ultraviolet-light emitting portion configured to emit ultraviolet light toward the media that are transported, the ultraviolet-light emitting portion being a first ultraviolet-light emitting portion, and
the control unit is configured to, when an elapsed period from a time when the medium accommodation portion is set in the attached state is a first period, cause a period, in which the second ultraviolet-light emitting portion emits ultraviolet light to the media, to be shorter than when the period is a second period that is shorter than the first period.

5. The recording device according to claim 1, wherein
the device body includes:
a stack height detector configured to detect a stack height of the media in the medium accommodation portion; and
a second ultraviolet-light emitting portion configured to emit ultraviolet light toward the media that are transported, the ultraviolet-light emitting portion being a first ultraviolet-light emitting portion, and
the control unit is configured to, when the stack height is a first height, cause a period, in which the second ultraviolet-light emitting portion emits ultraviolet light to the media, to be shorter than when the stack height is a second height that is lower than the first height.

6. The recording device according to claim 4 comprising
a feed tray for setting and feeding the media one by one, and
the control unit uses an equal period as an emission time in which the second ultraviolet-light emitting portion emits ultraviolet light to the media fed from the feed tray.

7. The recording device according to claim 1, wherein
the recording section includes a liquid discharging head configured to discharge a liquid to the media to perform recording,
a first transport region is provided downstream of the recording section in a transport path configured to transport the media, the first transport region being configured to transport the media at a reduced transport velocity of the media, and
a second ultraviolet-light emitting portion configured to emit ultraviolet light toward the media is provided in the first transport region, the ultraviolet-light emitting portion being a first ultraviolet-light emitting portion, and
the control unit controls the second ultraviolet-light emitting portion so as to emit ultraviolet light to the media that pass through the first transport region.

8. The recording device according to claim 1, wherein
a switch-back path is provided downstream of the recording section in a transport path configured to transport the media, the switch-back path being configured to perform switch-back transport of the media,
a second ultraviolet-light emitting portion configured to emit ultraviolet light toward the media is provided at an entrance to the switch-back path, the ultraviolet-light emitting portion being a first ultraviolet-light emitting portion, and
the control unit controls the second ultraviolet-light emitting portion so as to emit ultraviolet light to the media when the media enter the switch-back path and when the media exit the switch-back path.

9. The recording device according to claim 1, wherein
the recording section includes a liquid discharging head configured to discharge a liquid to the media to perform recording,
the liquid discharging head is provided so as to move in a width direction that is a direction intersecting a medium transport direction,
a second ultraviolet-light emitting portion configured to emit ultraviolet light toward the media is disposed at a position that is opposable to the media when the liquid is discharged by the liquid discharging head, the ultraviolet-light emitting portion being a first ultraviolet-light emitting portion, and
the control unit controls the second ultraviolet-light emitting portion so as to emit ultraviolet light to the media when the liquid is discharged by the liquid discharging head.

10. The recording device according to claim 1 comprising:
an attached state detector configured to detect an attached state of the medium accommodation portion, and
a control unit configured to control the ultraviolet-light emitting portion, and configured to, when an elapsed period from a time when the medium accommodation portion is set in the attached state is a first period, cause a period, in which the second ultraviolet-light emitting portion emits ultraviolet light to the media, to be shorter than when the period is a second period that is shorter than the first period.

11. The recording device according to claim 1, comprising:
a stack height detector configured to detect a stack height of the media in the medium accommodation portion, wherein
the control unit configured to control the ultraviolet-light emitting portion, and is configured to, when the stack height is a first height, cause a period, in which the ultraviolet-light emitting portion emits ultraviolet light to the media, to be shorter than when the stack height is a second height that is lower than the first height.

12. A recording device comprising:
a liquid discharging head configured to discharge a liquid to a medium to perform recording;
a first transport region disposed downstream of the recording section in a transport path configured to transport the medium, the first transport region being configured to transport the media at a reduced transport velocity of the media;
an ultraviolet-light emitting portion provided in the first transport region and configured to emit ultraviolet light toward the medium; and
a control unit configured to control the ultraviolet-light emitting portion and configured to control the ultraviolet-light emitting portion so as to emit ultraviolet light to the medium that passes through the first transport region.

13. A recording device comprising:
a recording section configured to perform recording on a medium;
a switch-back path disposed downstream of the recording section in a transport path configured to transport the medium, the switch-back path being configured to perform switch-back transport of the medium;
an ultraviolet-light emitting portion disposed at an entrance to the switch-back path and configured to emit ultraviolet light toward the medium; and
a control unit configured to control the ultraviolet-light emitting portion, the control unit being configured to control the ultraviolet-light emitting portion so as to emit ultraviolet light to the medium when the medium enters the switch-back path and when the medium exits the switch-back path.

14. The recording device according to claim 1 comprising:
a liquid discharging head configured to discharge a liquid to the media to perform recording, wherein
the liquid discharging head is provided movably in a width direction that is a direction intersecting a medium transport direction,
an ultraviolet-light emitting portion configured to emit ultraviolet light toward the media is disposed at a position that is opposable to the media when the liquid is discharged by the liquid discharging head, and
the control unit configured to control the ultraviolet-light emitting portion is configured to control the ultraviolet-light emitting portion so as to emit ultraviolet light to the media when the liquid is discharged by the liquid discharging head.

15. The recording device according to claim 1, wherein
when a jam occurs in a transport path configured to transport the media, the control unit stops emitting ultraviolet light to the media.

16. The recording device according to claim 1 comprising:
a cover configured to open and close to expose an interior of the device; and
an open-close detector configured to detect an open-close state of the cover, wherein,
upon detecting switching from a closed state of the cover to an opened state, the control unit stops emitting the ultraviolet light to the media.
